(19) United States
(12) Patent Application Publication
(10) Pub. No.: US 2022/0241786 A1
(43) Pub. Date: Aug. 4, 2022

(54) Centrifugal Microfluidic Chip, Kit and System for On-Chip Gas Supply

(71) Applicant: National Research Council of Canada, Ottawa (CA)

(72) Inventors: Lidija Malic, Saint Leonard (CA); Teodor VERES, Montreal (CA); Liviu CLIME, Longueuil (CA); Jamal DAOUD, Montreal (CA)

(73) Assignee: National Research Council of Canada, Ottawa, ON (CA)

(21) Appl. No.: 17/617,129

(22) PCT Filed: Jun. 26, 2020
(86) PCT No.: PCT/IB2020/056095

§ 371 Date: Dec. 7, 2021

Related U.S. Application Data
(60) Provisional application No. 62/867,931, filed on Jun. 28, 2019.

(51) Int. Cl. B01L 3/00

(52) U.S. Cl. CPC B01L 3/502753; B01L 2200/027; B01L 2300/123; B01L 2400/06; B01L 2400/0487; B01L 2200/0689

(57) ABSTRACT

A centrifugal microfluidic chip is provided that allows an on-chip chamber to provide humidification control, or more generally, gas composition control, to another chamber of the chip. This allows for microfluidic incubation using low-cost and efficient centrifugal devices such as multi-port pneumatic chip controllers, single or multi-port pneumatic slip rings, and articulated centrifugal blades with a pneumatic slip ring. The device may be used for cell culturing, microorganism testing, or production of chemical species from biological samples with a controlled microenvironment.

### FIELD OF THE INVENTION

The present invention relates in general to controlled gas supply to chambers of a centrifugal microfluidic chip, and in particular to a chip having a conditioned chamber, and 3 reservoirs coupled thereto in a layout that allows one reservoir to supply a gas to the conditioned chamber, another to supply a liquid, and a third to receive output from the conditioned chamber.

### BACKGROUND OF THE INVENTION

There are many trite virtues of microfluidic processing. Parsimonious use of samples and reagents, and testing/reacting/culturing in a very small space, are some. Many applications require control over composition, and possibly (temperature, and/or pressure) of both liquid and solid phases, of a conditioned chamber. For example, in microbiological testing, production, or reactor chambers, a microorganism (cell, organelle, bacteria, viruses, archaea, fungi, protozoa, organoid, or small tissue biopsy), or food or aqueous sample potentially containing any of the above, in the conditioned chamber may be supplied liquids (nutrients, catalysts, or reactants) while also controlling composition, temperature and pressure of gas, to treat, test, process, or incubate the microorganism.

As such, conditioned chambers are needed in fundamental research (cell biology, biochemistry, physiology, ecology, evolution), as well as cellular production of difficult to synthesize species by microorganisms. Specifically automating and integrating cell-based assays is needed for drug screening, clinical diagnosis and cell-based therapy.

Traditional microtiter plate methods are labor-intensive and difficult to automate without the use of large and expensive robotic liquid handling systems. A variety of lab on chip microfluidic systems have been developed to facilitate manipulation of very low fluid volumes thus successfully miniaturizing cell culture assays.

Many microfluidic systems have been developed over the past two decades in order to overcome some of the aforementioned problems, and allow continuous cell culture and incubation, while integrating cell trapping, cell-based assays and detection [Halldorsson, S., Lucumi, E., Gómez-Sjöberg, R. & Fleming, R. M. T. Advantages and challenges of microfluidic cell culture in polydimethylsiloxane devices. Biosens. Bioelectron. 63, 218-231 (2015)]. Microfluidic systems miniaturize cell cultures, reduce reagent consumption, and thus the overall cost of the assay. They also have the ability to reduce the ratio of extracellular to intracellular fluid volumes allowing precise manipulation of cellular microenvironment to decrease the lag in cell response to external stimuli and thereby reduce assay time [Kane, K. I. W. et al. Automated microfluidic cell culture of stem cell derived dopaminergic neurons in Parkinson's disease. bioRxiv 209957 (2017)]. Moreover, miniaturized cell culture allows multiplexing within a small geometrically confined footprint allowing experimental replicates or screening of multiple conditions in parallel [Reichen, M., Veraitch, F. S. & Szita, N. Development of a Multiplexed Microfluidic Platform for the Automated Cultivation of Embryonic Stem Cells. J. Lab. Autom. 18, 519-529 (2013)]. Finally, as the microfluidic devices possess inherently closed channels and chambers for fluid manipulation, they can minimize effects of evaporation while allowing continuous perfusion of cell culture media and nutrient and stimulant delivery [Nakatani, E. et al. Compartmentalized microfluidic perfusion system to culture human induced pluripotent stem cell aggregates. J. Biosci. Bioeng. 124, 234-241 (2017); Khoury, M. et al. A microfluidic traps system supporting prolonged culture of human embryonic stem cells aggregates. Biomed. Microdevices 12, 1001-1008 (2010)].

Despite of these developments, some of the advantages of microfluidics have yet to be realized, as gas-phase conditioning using prior art microfluidic devices involve using permeable microfluidic chips that exchange gasses readily with an ambience of the chip. This leads directly to requiring that these chips be placed in large auxiliary equipment (incubators, with syringe pumps, etc.). Indeed, most of the microfluidic systems described in the literature rely on the use of external syringe pumps to supply CO₂ buffered media to the culture chambers, adding to the overall complexity of the device operation and limiting their practical application [Kyu Byun, C., Abi-Samra, K., Cho, Y.-K. & Takayama, S. Pumps for microfluidic cell culture. Electrophoresis 35, 245-257 (2014); Takano, A., Tanaka, M. & Futai, N. On-chip CO₂ incubation for pocket-sized microfluidic cell culture. Microfluid. Nanofluidics 12, 907-915 (2012)]. Moreover, these devices are fabricated using PDMS, owing to its transparency, biocompatibility and gas permeability allowing control of the gaseous microenvironment [Torino, S. et al. PDMS-Based Microfluidic Devices for Cell Culture. Inventions 3, 65 (2018)]. While PDMS devices are abundant in academic research, the material is incompatible with scalable manufacturing and is rarely used in industry, including pharmaceutical and clinical research where biocompatible hard thermoplastics such as PS and COC prevail. Hard thermoplastics have gas permeabilities orders of magnitude lower than PDMS. In addition, PDMS can absorb proteins and small molecules, biasing some assay results. Gas permeability of PDMS can lead to sample evaporation over time, unless the chip is in a humidifier chamber. Applications requiring prolonged cell incubation invariably require humidity control. Indeed, most of the experiments in literature use PDMS chips within humidified cell culture incubators, reducing the “lab on a chip” to a “chip in a lab”.

Prior art researchers struggling with this precise issue, for example Bunge, F., van den Driesche, S. & Vellekoop, M. J. PDMS-free microfluidic cell culture with integrated gas supply through a porous membrane of anodized aluminum oxide. Biomed. Microdevices 20, 98 (2018), were motivated to provide an improved gas permeable medium for supporting chips in incubators, for growing cells.

Prior art chips, that may not be centrifugal microfluidic chips, but may have some or several structural features in common with the present claims, are: US 2009/246082, WO 2018/215777, US 2018/364270, US 2017/173589, US 2016/214105, US 2008/226504, US 2018/313765, JP 2003344421, EP 2332653, CN 107460122, U.S. Pat. No. 7,452,726, and U.S. Pat. No. 10,252,267.

There is therefore a need for a centrifugal microfluidic chip that is compact, and designed to permit liquid alimentation and gas supply control within a conditioned chamber thereof, preferably with few limits on material composition of the chip (e.g. compatible with mass manufacturing techniques, inert, low cost forming and sealing, etc.). Particularly a chip that allows for direct control over gas supply to a conditioned chamber of a centrifugal microfluidic chip, without passing through a permeable membrane and therefore subject to absorption and desorption of other volatiles.

### SUMMARY OF THE INVENTION

While it is very common in microfluidic chips of all types to provide a chamber, possibly with cell or microorganism support structures in the chamber, that is connected with: one or more liquid supply reservoirs for alimenting or perfusing the chamber; and one or more waste reservoirs for receiving fluid from the waste reservoirs, the idea of adapting a microfluidic reservoir to serve as an on-chip controller to supply a gas for a conditioned chamber, particularly in centrifugal microfluidic contexts with pneumatic control, was not known. This is a surprisingly elegant solution to the problem of how to control H₂O, CO₂, O₂, N₂, CH₂, CO, CH₃ etc. that obviates passage through a membrane.

The solution involves providing a gas supply (GS) reservoir, coupled to the conditioned chamber (CC), in a manner that is peculiar for centrifugal microfluidic devices. The coupling is through a channel, where the channel passes closer to a reference axis for the chip than either the GS reservoir or CC. This makes the channel generally unsuited to, and needlessly problematic for, conducting liquids therebetween. However, it makes an excellent barrier for liquids, and poses almost no barrier for gases. Thus a reactive liquid or solid precursor, or volatile liquid in the GS reservoir, will itself not be movable into the CC, but the gas products can be. Generation of the gas may be controlled externally by controlling a temperature of the GS reservoir. Coordinating this generation with a controlled flow rate from a port of the GS reservoir, through the channel, to the CC, and out of the chip via a CC port, controls the CC gas concentration.

To control the flow rate, both the GS port and opening to the channel are above a fill line of the GS reservoir, as this precludes entrainment of the liquid into the channel. Herein a fill line is understood to be a free surface of a liquid content of the chamber/reservoir when “full” give or take a meniscus of the fluid. A chamber, of course, can be overfilled, which may make it unsuited to a specific protocol, or operation, or may make it unusable entirely. While a fill line is not usually an indelible marking of an unfilled chip 1—it may in fact be demarcated; 2—it may be identifiable in a product by a stated volume for the chamber of a chip in instructions supplied by a chip vendor; or 3—it may be evident by examination having regard to the following cues: a) positions of the GS port and opening to the channel; b) positions of all other functionally connected reservoirs relative to their inlets, outlets and ports; c) positions of apparatus for supporting a material within the CC, such as a cell support, which is naturally assumed to support the material at or below the fill line. Note that a fill line is geometrically not a line, but is defined by a centrifugal field that radiates from an axis of rotation of the chip. It should be noted that if the chip is designed for “on edge” rotation, i.e. a top edge of the chip is parallel with an axis of the centrifuge, the first arc is in a thickness direction of the chamber and can be essentially neglected, resulting in all chambers having essentially parallel geometric lines as fill lines. The other likely orientation of chips is with the axis perpendicular, and offset from a normal of the chip's surface, in which case the fill lines of all chambers are essentially respective arcs of circles from the same axis.

While the axis of rotation may or may not be defined, just looking at the chip, there are cues that provide an operable range of positions for the axis, consistent with a functional view of the chambers thereof, given the channel interconnections and positions of the channels with respect to the chamber. Thus a holistic, purposive view of a chip will, in almost all cases with 4 or more chambers arrayed and interconnected for a functional result, provide a narrow range of possible axes of rotation, and define, within a narrow band, a fill line for each chamber. Further reasonable limitations, such as the fact that a chip's axis is never so far away from the chip itself as to greatly decrease a centrifugal field gradient applied at the chip, and increase a moment on the centrifuge, as to require a higher torque to achieve lower gradient. As such, the axis of the chip is generally expected to be separated from the chip by less than twice a length of the chip.

The present invention also includes possibility of the chip being designed to tilt on an axis parallel to the axis of rotation, such as taught in Applicant's co-pending WO 2015/181725 or in the background thereof (the contents of which are incorporated herein by reference where permitted by law and practice, and presumed known in the art in all other jurisdictions). If so, the fill lines of the chambers refer to those at a baseline pose of the chip, if one exists, or a balanced pose at a mid operational range. Such chips are identifiable by use of non-capillary driven, serpentine channels that retain or dispense fluid depending on the tilt angle.

The channel may couple GS reservoir to the CC either above or below the fill line of the CC. By supplying gas below the fill line, the gas will dissolve into the liquid content of the CC more efficiently, and a higher pressure is required to “bubble” the gas into the CC, compared to gaseous delivery to the CC above its fill line. Bubbling can advantageously mix CC content, or impede cell attachment or settling as desired in culture of some cells. Bubbling is taught in Applicant's co-pending WO 2015/132743 and in the prior art section thereof, which also teaches a preferred multi-channel pneumatic control architecture for centrifugal microfluidics (the contents of which are the contents of which are incorporated herein by reference where permitted by law and practice, and presumed known in the art in all other jurisdictions).

To encourage diffusion of gas into a stream at the GS reservoir, or from the stream into the CC's content (particularly when bubbling is not used), a surface area of the contact with the gas (fill line) may be enlarged. A width of the CC or GS reservoir may be greatest at or near their respective fill line. To greater effect, an etch depth of the CC and/or GS reservoir may be substantially greater than that of the channels, and some other reservoirs of the chip. A candidate reference axis position may be more or less likely depending on the fill line surface area of the CC and GS reservoirs relative to those of other candidates.

In order to reasonably expect that the chip's fill lines can be ascertained by inspection; and to make a chip that is functional for a variety of applications, the chip is limited to the CC coupled at least to a first buffer or reagent supply (SUP) reservoir, and a first output (OUT) reservoir, such as a conventional waste reservoir, supernatant, or centrifugally isolated fraction of the CC. In most embodiments, two or more SUP and OUT reservoirs are preferred. The SUP reservoir is coupled to the CC via a SUP channel that meets the SUP reservoir below its fill line, and preferably at a distal surface of the SUP reservoir, relative to the axis. The SUP channel may couple to the CC anywhere above the CC fill line. The OUT reservoir is coupled to the CC by an OUT channel, which meets the OUT reservoir above a fill line, and the CC below the fill line, with preference to the collected sample. For example, if the OUT reservoir has a small volume relative to a fill volume of the CC, it may be designed to skim a top surface of the fill volume, after/during some amount of centrifugation, or otherwise extract a different fraction from a corresponding point intermediate the CC's distal wall and fill line.

Some applications call for delivery of a substantial volume of gas, or the gas delivery may be inefficient, and delivered over a long period of time. Cell incubation studies may take many hours or several days, for instance. If so, a lot of gas has to be produced with the material below the fill line of the GS reservoir: and a higher fill volume of the GS reservoir is desirable. Spatial constraints on centrifugal microfluidic chip design generally require a trade-off between fill volume, while providing adequate surface area at the fill line, and also allowing for a gradual loss of the free surface area as the GS reservoir empties, so that concentration drops in the stream during use are not extreme.

As such, the chip is used by mounting it to a centrifuge, and coupling it with a system that is adapted to supply the controlled flow rate into the GS port and out of the chip via a CC port, such as the systems taught in Applicant's WO 2015/132743 and prior art identified therein, including single channel pneumatic slip rings.

WO 2015/132743 teaches a centrifugal chip controller with programmable electromechanical valves on the rotating stage, making it possible to apply regulated air pressure to dedicated pressure ports of the chip through a pneumatic interface, and an electronic controller of the valves for directing the operation of the valves. Each pressure port can be programmed to apply either positive or negative pressure from the pump or normal atmospheric pressure (vent). The pump can be connected to a gas supply, such as gas cylinder in order to provide specific gas environment to the cartridge, such as CO₂ required for cell culture, or to a pump which supplies air. Pressure differences generated using the centrifugal chip controller allow performance of a variety of fluidic functions such as valving, flow switching, reverse pumping (moving fluid against centrifugal force), or on-demand bubble-based mixing without the need for integrating any active element on the cartridge.

A copy of the claims are incorporated herein by reference.

Further features of the invention will be described or will become apparent in the course of the following detailed description.

### BRIEF DESCRIPTION OF THE DRAWINGS

In order that the invention may be more clearly understood, embodiments thereof will now be described in detail by way of example, with reference to the accompanying drawings, in which:

FIG. 1 is a schematic illustration of a centrifugal microfluidic chip offered as an embodiment of the present invention, adapted for use with a multi-port pneumatically controlled microfluidic chip controller;

FIG. 2 is a variant of the chip of FIG. 1 with an alternative pneumatic valving arrangement in one channel, and illustrating limits on reference axis positions of the chip;

FIG. 3 is a variant of the chip of FIG. 1 adapted for use with a single port pneumatically controlled microfluidic chip controller or a pneumatic slip ring with an articulated blade holder that provides for a pivoting of the chip on an axis perpendicular to the chip surface;

FIG. 4 is a variant of the chip of FIG. 1 adapted for use with a single port pneumatically controlled microfluidic chip controller or a pneumatic slip ring with a hydrodynamic controlled metering and dispensing system;

FIG. 5 is a schematic illustration of a conditioned chamber of a chip or variant offered as an embodiment of the present invention, in which microorganism support structures are provided below a fill line of the conditioned chamber;

FIG. 6 is a variant of the embodiment of FIG. 5 in which a gas trap is provided to increase a diffusive efficiency of the gas delivered into the conditioned chamber; and

FIGS. 7A-E are photographs showing a prototype chip used in demonstrating the present invention, respectively showing layouts of 3 patterned chip surfaces of two pieces of a cartridge used to demonstrate the present invention, and a filled cartridge before and after an incubation process.

### DESCRIPTION OF PREFERRED EMBODIMENTS

Herein a centrifugal microfluidic chip is described that has particular value for use in processes that call for conditioning of a chamber with a supply of gas of a given concentration, as well as possibly pressure and temperature. To avoid possible contamination issues, or requirements for off-chip supply, and to simplify the microfluidic system, the gas supply is adapted to be provided from a single reservoir of the chip, which is adapted to contain a volatile or otherwise gas-productive liquid volume below its fill line. By locally heating the liquid volume, a direct supply of the gas can be provided on chip, avoiding any condensation or separation issues that may arise if the gas were supplied directly through a pneumatic slip ring, or other path that crosses substantial temperature gradients.

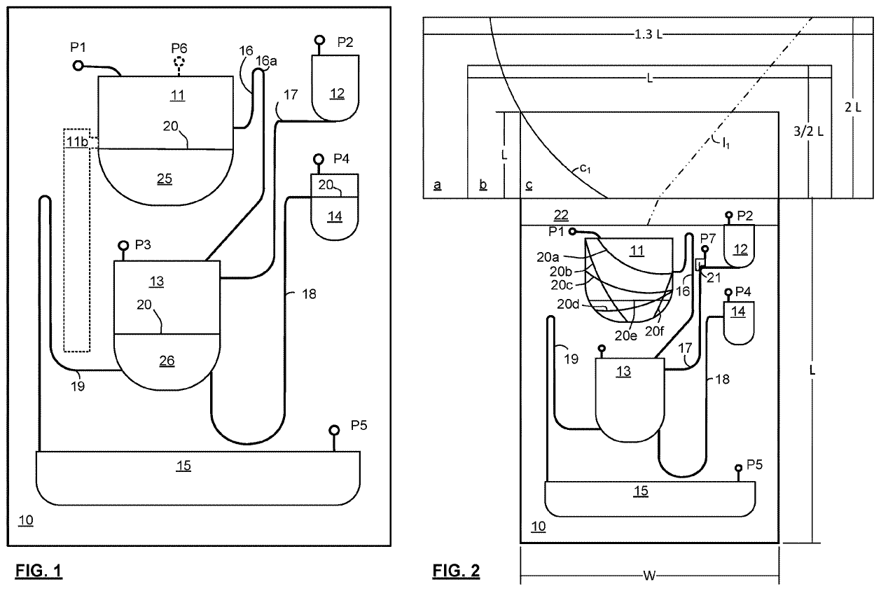

FIG. 1 illustrates a first embodiment of a centrifugal microfluidic chip 10 in accordance with an embodiment of the present invention. This embodiment is particularly suited to use with a centrifuge system having multiple (5 or 6) independently (i.e. any set of ports can be controlled at any time) or selectively (i.e. any one can be controlled, but only one at a time) controlled ports of the chip 10, such as provided according to the teachings of Applicant's co-pending WO 2015/132743. While the ports are shown local to respective chambers, in general there is little disadvantage to having all of the ports arrayed along a common edge of the chip 10, for easy alignment, and may be part of a common interface design for the chip, to obviate manual coupling of individual tubes.

Chip 10 has a conditioned chamber (CC) 13, which may be for cell culture or tissue growth, or for growth or testing of a live tissue, organelle, microorganism, or sample. The CC 13 has a number of reservoirs fluidically coupled thereto, including: a supply (SUP) reservoir 12, outlet (OUT) reservoir 14/15, and a gas supply (GS) reservoir 11. Each reservoir 12,14/15,11 has a respective channel (SUP 17, OUT 18/19, GS 16) for coupling to the CC 13. Each reservoir has a respective port (P2, P4/P5, P1) for liquid loading, as does the CC 13 (P3). The same port can also be used for vent or to individually address each chamber with a pneumatic source in order to apply positive or negative pressure, as required by an assay protocol or microfluidic process. GS reservoir 11 may have a separate port P6 for supplying a volatile liquid as well as the port P1 for coupling to a pneumatic source, such that the GS reservoir 11 can be continuously replenished without interrupting humidification. This may be performed by off-chip loading as taught in WO 2015/132743. Preferably the port P6 is plugged with the volatile liquid in use, so that it offers no exhaust from GS reservoir 11.

The port P6, or optionally a SUP reservoir 12, can also be supplied by a stationary, non-contact, drip delivery system as taught in Applicant's co-pending U.S. 62/760,256 WORLD-TO-CHIP AUTOMATED INTERFACE FOR CENTRIFUGAL MICROFLUIDIC PLATFORMS. As GS reservoir 11 is a pressurized chamber, some attention to independent control over the rates of flow of both the carrier gas of P1 and liquid of P7, as well as evaporation losses through P1. In particular this may be accomplished by blocking P7 with a liquid plug, and providing a substantial hydrodynamic resistance or a valve at the opening of P7. For SUP reservoir 12, P2 may be used without any need to mitigate dual flow issues or maintain pressurization.

A GS reservoir (11) supplies a gas to the CC 13, for example to maintain a humidity of the CC 13 throughout a cell incubation or growth process, or otherwise control a gas composition within the CC 13 above the fill line. GS reservoir 11 is connected via the GS channel 16 to the CC 13, either below or above a fill line 20 of the CC 13. If below the fill line, a higher pressure must be supplied at the port P1 to push the gas through liquid content of the CC 13 (resulting in “gas bubbling”). An advantage of this is the high surface area of the bubbles that leads to a higher dissolution of the gas within the liquid. If the gas output from the GS reservoir 11 has a higher dissolution rate than a carrier gas providing the pressurized flow from P1 to P3, a higher efficiency delivery of the gas can be effected. Bubbling can abet mixing and avoid sedimentation or attachment of microorganisms or cells. Mixing vortices may be disadvantageous for some cell cultures, and these may be avoided with barriers protecting cell scaffolds/supports from the bubbles, and directing the bubbles away from the cells. Alternatively, as shown, diffusion between a free surface of liquid content of the CC 13 (at or below the fill line 20) may be relied upon for supplying the gas with the liquid contents.

The channel 16 can meet the GS reservoir 11 anywhere above a fill line 20 of the GS reservoir 11, either on a top or on a side thereof. In principle, the channel 15 could meet the GS reservoir 11 below the fill line 20 to achieve a similar advantage in terms of efficient gas entrainment, however safeguards would be needed to prevent liquid occlusion of the channel 16 during the bubbling, as obstructions in this path would be inconvenient, and bubbling of most liquids, such as aqueous liquids, are likely to produce these obstructions. GS channel 16 is a serpentine channel, which may have a high hydrodynamic resistance or substantially none, as the channel 16 is not intended to conduct a liquid, but it supplies a minimum resistance to gaseous transport. If a high hydrodynamic resistance is provided, it may prevent or reduce risk of liquid entering the channel 16 during loading, which might be performed prior to centrifugation. If it has low hydrodynamic resistance, any temporary blockage of the channel 16 may be cleared with less pressure and time. Accordingly, the GS channel 16 may have a lower hydrodynamic resistance except near the opening to the GS reservoir 11.

The GS channel 16 defines a serpentine structure similar to syphon valves, well known in the art, but does not necessarily have most issues associated with syphon valves: the channel 16 does not have any particular hydrophilicity, or hydrodynamic resistance that are essential for reliable operation of syphon valves. As such, exacting dimensional control, and surface functionalization are unnecessary. But the serpentine path includes a segment 16a that is closer to the chip's axis of revolution, or a reference thereof, than the GS reservoir 11 or the CC 13. Having regard to the fill lines 20 shown in GS reservoir 11 and the CC 13, which are parallel lines, the axis is inferably parallel to a top edge of the chip 10, although, as will be shown and explained in reference to FIG. 2, the chip 10 could be equally deployed for revolution about an axis positioned in a range of positions that would affect a shape of the fill lines. This, along with positions of the openings to the port P1 and channel 16, guards against liquid from the GS reservoir 11 being driven under centrifugal force into the CC 13.

SUP channel 17 extends from an axis-distal point of the SUP reservoir 12, to the CC 13, above fill line 20. Port P2 extends from an axis-proximal point of the SUP reservoir 12. As such the fill line of reservoir 12 may be a top edge of the reservoir. There is no risk of overfilling SUP reservoir 12. SUP channel 17 is preferably a channel with a low hydrodynamic resistance, and regardless, once primed, is continuously subjected to a negative pressure (relative to the CC 13) to avoid rapid dispensing of it's liquid content under the centrifugation.

OUT channels 18,19 are both shown extending from the CC 13, below the fill line 20, to respective OUT reservoirs. OUT reservoir 14 is for a supernatant, and has a specific position with respect to the fill line that is associated with desired centrifugation properties (typically above a position where cell detritus and particulates may collect during high rate centrifugation), but low enough to collect a desired volume of the supernatant. The OUT channel 18 meets reservoir 14 at a fill line 20 thereof. As OUT reservoir 14 is axis-proximal the fill line of CC 13, the only way to draw the supernatant into it is with reverse pumping: applying a pressure at P4 that is sufficiently negative with respect to the CC 13, to overcome the inertia of the supernatant. If the supernatant chamber is overfilled, simply releasing the pressure at P4 while under centrifugation, will ensure that the excess liquid will return to the CC 13. As such channel 18 is preferably a low hydrodynamic channel.

OUT channel 19 leads to an axis-proximal point of a waste reservoir 15. As OUT reservoir 15 is axis-distal the CC 13, and the channel 19 is of low hydrodynamic resistance, excess liquid contents can be extracted, to make room for fresh buffer for example, by decreasing a pressure at P5 (relative to CC 13) until channel 19 is primed, and then increasing the pressure once the excess liquid is extracted, to prevent emptying of the CC. Once the liquid in OUT channel 19 retracts above the syphon, pressure at P5 can be released, and the liquid will fall back into CC 13.

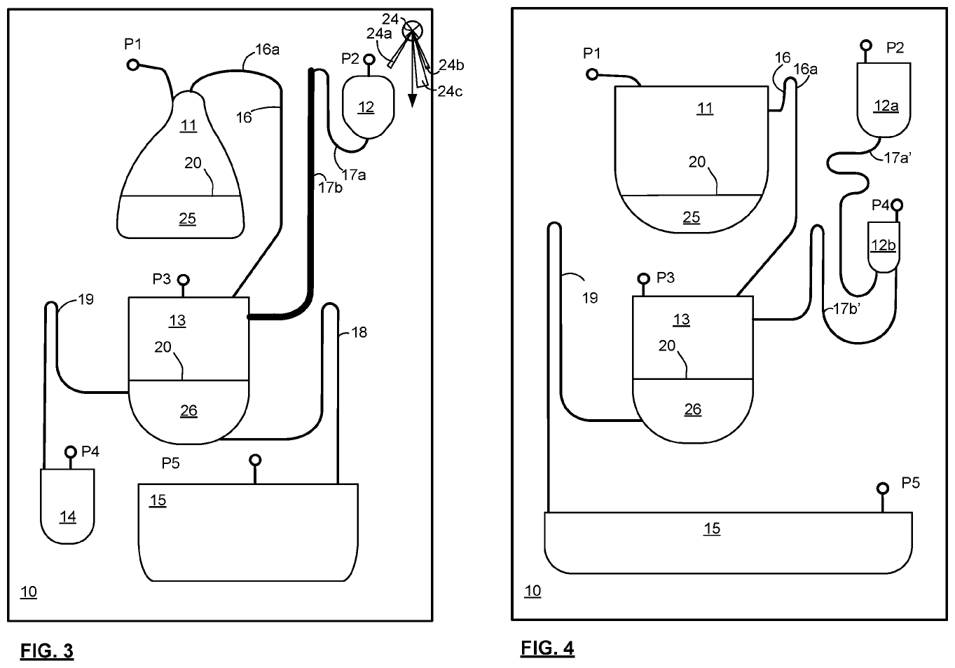

FIG. 1 also illustrates a spillway 11b which is optionally incorporated into GS reservoir 11, and is not essential to the invention in all characterizations. A spillway 11b is a readily identifiable marker of fill level of a chamber. The spillway could additionally or alternatively be included in the CC 13, to avoid overfilling. Spillways may be particularly preferred in embodiments designed for use in articulated centrifugal blades, such as described herein below with respect to FIG. 3, as operation of articulated centrifugal blades may be particularly sensitive to a fill level of the chambers.

A principle advantage of the spillway 11b is that it prevents over filling of GS reservoir 11 from impacting functioning of the chip 10. If the volatile fluid is supplied into GS reservoir 11 prior to centrifugation, and it is not desired or convenient to provide high accuracy metering of the volatile fluid, or further if the volatile fluid is supplied continuously at a rate that is not controlled with sufficient accuracy relative to the evaporation rate, the spillway 11b will draft any excess fluid into an adjacent reservoir, as soon as/while centrifugation is applied. Thus a minor error in the fill volume can be accommodated without risk of occluding the GS channel 16. The use of a spillway 11b therefore increases a volume of volatile liquid that can be used without increasing risk of occlusion, or requiring careful metering of the volatile liquid 25.

There are several properties of the chip that are arbitrarily represented. A size, shape, orientation and layout (relative positions) of each reservoir/chamber is not required to be as shown. In general, the shape of the GS reservoir 11 preferably provides a low variation of a surface area of a free surface of liquid content when the liquid occupies between 20% and 100% of the volume below the fill line 20. This ensures that, as a volume in the GS reservoir 11 drops from gas production that is drawn into CC 13, a rate of gas production and entrainment, does not appreciably vary. Furthermore a relatively high free surface area may be preferred, such that the GS reservoir 11 may be a deep-etched structure of the chip 10, and may occupy a larger surface area than other chambers. The GS reservoir 11 is shown axis-proximal CC 13, but this can be reversed, or they could be equally spaced from the axis.

The CC 13 is shown relatively large, and also preferably deep, also to provide a high free surface area of the liquid content. However, if the GS channel 16 meets the CC 13 below the fill level, the CC needs much less volume above the fill line, avoiding free surface area constraints. Depending on the processes for which the CC 13 is designed, it may have a number of different features. It may have cell, microorganism, or tissue traps, scaffolds or supports entirely below the fill line, or may provide cell culturing at the free surface.

During chip operation it may be desired to independently control a pressure and temperature within the CC 13. Temperature control can be accomplished with various on-chip and off-chip heating systems well-known in the art. Many techniques for heating in microfluidic devices have been described in literature [V. Miralles, A. Huerre, F. Malloggi and M.-C. Jullien, A Review of Heating and Temperature Control in Microfluidic Systems: Techniques and Applications, 2013, vol. 3.]. Off-chip heating techniques include Peltier elements, resistive elements, laser diodes (argon-ion laser, infrared laser), etc. On-chip techniques include integrated microheaters using thin metallization layers (gold, platinum, copper, chrome), liquid metal embedded in microchannels as resistive elements, and miniaturized microwave heating elements. In accordance with this desire, a coating or embedded material, such as a metal, can be applied within CC 13 (and optionally also reservoirs 12,14) to assist in heat absorption, retention, and distribution across a volume to be heated. This volume may at least 60% align with the CC 13, or the CC 13 below the fill line, or in addition thereto, one or more SUP reservoirs, and preferably excludes any part of the GS reservoir 11, to permit independent thermal control of the GS reservoir 11 and the CC 13. Instead of applying heating from within the chip, the absorbing and conducting material may be applied on a back of the chip, or may be integrated with the material of the chip. If the latter, the material is preferably at least ten times more absorbing around the volume, than it is elsewhere on the chip (away from the GS reservoir 11), such that application of heat by a laser, diode, eddy current, or like source across an annular strip of the chip 10, selectively heats one of these independently controlled thermal zones. Finally the material may be provided on a support for the chip in line with an intended mounting position for the chip. To provide higher accuracy temperature control, some attention to a spacing between the material and chip should be controlled, for example via a thermal coupling fluid or clamp.

Pressure control can be exerted, as well as throughput of the gas, by controlling pressure at all of the ports concurrently, and providing no free-vented chip ports, to within pressurization limits of the chip. Likewise pressure variation can be supplied to the CC 13 by pulsing pressure supplied at ports.

For cell culturing, or several other biological processes, heating at 35-40° C., and more preferably 36-39° C., 37-38° C. or about 37.5° C. is ideal for CC 13. Control over CO₂, or humidity of an air stream through GS reservoir 11 may conveniently involve controlling temperature between room temperature and 35-40° C. as well. One of the applications of this invention is to humidify air that passes across the CC 13, to prevent evaporation losses in a warm chamber containing an aqueous liquid that requires gas exchange, as is necessary in many biological sample studies. By heating water in the GS reservoir 11, and limiting a carrier gas flow rate, the stream passing through CC 13 above the fill line 20, has a high enough humidity to greatly reduce evaporation losses.

The chip 10, mounted to a centrifuge by a suitable centrifugal microfluidic controller, can perform a variety of function, while centrifugation is continuous. For example, once a biological sample is loaded into CC 13, and GS reservoir 11 and SUP reservoir(s) 12 are loaded, the centrifugation may begin. While the centrifugation rate is above a threshold, liquid in each of the reservoirs will be at or below respective fill lines. On demand, or according to a scheduled protocol, a pneumatically actuated positive pressure (relative to CC 13) can be supplied at port P2 to transfer some fluid from the SUP reservoir 12 to CC 13. Drip-based metering of the media transferred to CC 13 can be achieved by applying short positive pressure pulses intermediate negative pressures at P2 at pre-determined intervals. Alternatively, all the media can be transferred at once, as per assay requirements.

The chip 10 may be centrifuged at different rates, at different points of time. For example, a high rate may be used to sediment cells to a bottom of the CC 13, or a support therefor, or post lysing to separate different structures; and a lower rate may be used during an incubation period. A liquid content from the CC 13 can be transferred to waste 15 through siphon channel 19 by applying a negative pressure at the port P5; or to supernatant chamber 14 through channel 18 (with negative pressure at the port P4).

The chip 10 may be supplied as a part of a kit with one or more of the following: fluid supplies, such as volatile liquid content 25 for GS reservoir 11, a liquid containing or potentially containing a biological sample 26 for CC 13, one or more reagents, buffers, solutions for SUP reservoir(s) 14; one or more cartridge forming elements that combined with chip 10 form cartridges that are readily coupled to a chip controller, directly to a centrifuge blade, to an articulated blade, or to a centrifuge with a pneumatic slip ring; a material for application to a cartridge, the chip, or a chip support, the material dimensioned to provide thermal control over one of the GS reservoir 11, CC 13, or a part of one of these below the fill line. Specifically the chip 10 containing the fluids is a microfluidic system that is also illustrated in FIG. 1. This chip 10 can have a cover lid, cartridge, or other structural elements for facilitating coupling of the ports P1-P7 to pressure supply lines of the chip controller or pneumatic slip ring.

FIG. 2 is a schematic illustration of a variant of the chip of FIG. 1 showing three preferred regions where an axis of rotation of the chip would be expected, and 5 specific fill lines that result from selection of the axis. Herein like reference numerals associated with features of different variants and embodiments of the invention, identify like features and their descriptions are not repeated herein unless to note a difference.

The variant of FIG. 2 differs only as shown from that of D1 in that port P7 is provided to control transfer via pneumatic valve 21, instead of P2. As such P2 is a vent or loading path for the chip (akin to P6), but is not needed for metering or selectively transferring liquid from SUP reservoir 12 to CC 13. Typically pneumatic valves can be embedded in chips that are composed of a TPE, or other elastomeric material. While TPEs may have higher gas permeabilities that thermoplastics, especially if thin, their permeabilities are typically far less than PDMS, and typically provide satisfactory gas barriers as produced. It may be useful to coat with vapour barriers, either inside the microfluidic chambers (at least 11 and 13), or over a back surface, to allow these chips to be used in the present invention. While chip 10 of FIG. 1 may be composed of a non-porous or low gas permeability, patterned film, FIG. 2, with valve 21, is softer. Advantages TPEs in forming and bonding chips to form devices are explained in Applicant's U.S. Pat. No. 9,238,346, and an advantage of embedded TPE valves include a simpler process for controlling transfer and metering.

P7 requires an active pressure source suited to actuating the valve 21. The valve 21 may be a normally closed valve, a normally open valve, or tristate valve with open, closed, and semipermanently closed states. The pneumatic valve may be as taught in Applicant's (U.S. Pat. No. 9,435,490, PCT/IB2019/051731, U.S. Pat. No. 9,238,346). Note a separation of a pressure manifold of the valve 21 is somewhat schematically shown, it is far closer to channel 17 than to channel 16, and therefore channel 16 has no valve in it, and is substantially unaffected by pressurization of this manifold.

While only channel 17 is shown controlled by a valve in FIG. 2, in alternate variants channels 18 and 19 are valved. If a large number of valves are used, layout can be simplified by providing a parallel pneumatic control layer, as is conventional in the art.

Preferably a reference axis position of the chip 10 is positioned within or above a top edge band 22 of the chip 10, which extends as a rectangle from the top edge to a chamber that is proximal the top edge. Typically a centrifugal microfluidics, chips have a length (L) of 3-20 cm (most commonly 4-18 cm, 4-8 or 12-18 cm, or about 5, 10 or 15 cm), and the reference axis position is within 1-5 cm of the top edge. However, if the chip has a centrifugally mounted controller, it may have machinery that displaces the chip from this axis, and as a result may be within the top edge band or a first box, a, twice L high by 1.3 L wide on the top edge of chip 10, centred with respect to the chip. More preferably the axis lies within the band 22 or a second box, b, that is 1.5 L high by L wide on the top edge, centred. Most preferably the axis lies within the band 22 or a third box, c, that is covered by a translation of the chip 10 so a bottom edge of the translation meets the top edge of the original chip position. Note that a scaling of the boxes is vertically compressed to facilitate viewing.

While boxes a,b,c delimit spaces within which the axis is positioned, the chip 10 specifically has a cue to the axis reference position. The first cue is found by the openings of the GS reservoir 10 to port P1, and channel 16. These two points have a geometrical perpendicular bisector I₁, and the optimal location for the axis reference position lies on I₁. This optimal location corresponds to a highest fill volume that does not block either opening. A highest volume is generally desired if an extended incubation period is required, for example, and continuous replenishment (e.g. via P6) of a volatile liquid in GS reservoir 10 is to be avoided. In other contexts an efficiency of gas delivery may be the primary concern. For some applications, a preference for a subset of each box a,b,c or strip 22 bounded by lines passing through the midpoint of the two points, at angles of +/−45°, more preferably +/−30°, +/−20°, +/−15°, +/−12° and +/−10° from

The effects of the reference axis location on fill lines is shown with a sampling of fill lines 20a-f. Note a refraction of I, is an artifact of the scaling of the boxes, and each fill line shown herein is shown by an arc of circle from a reference axis, without correction caused by meniscus—as such the free surface of the liquid will not exactly match the fill line as drawn. Fill line 20a shows a fill line at an optimal, preferred reference axis position, which is at a centre of the top edge of the chip (where line I₁ refracts). Formally, minutely higher fill volumes can be achieved with increasing distance to the axis, but 20a is a preferred point on the line because the closer the chip is to the axis, the higher the centrifugal field and the lower the moment on the centrifuge's blade. While fill line 20a is ideal, each of fill lines 20b-e shows alternatives that afford reasonably high volumes below the fill line. Two features of each fill line correspond to two parameters of the reference axis position that is associated with the fill line: a curvature of the fill line determines a distance of the fill line to the axis (e.g. 20e has a reference axis position 2L above the top edge, centred on the chip, whereas 20d's reference axis position is at the top edge); and a perpendicular bisector of any two points on the fill line passes through the axis (thus 20b's reference axis is to the right of the chip, 20d's is to the left of chip).

Each of fill lines 20b-e are shown well below their maximum fill lines so that the drawing can be clearly seen (if all were shown at the maximum fill line, the lines would be difficult to differentiate). Thus each of fill lines 20a,c,d,e clearly admits of a concentric fill line with a fill volume in excess of 60% of a volume of GS reservoir 11. Fill line 20b has a reference axis position at the limit of box a on the right side. This axis position can admit a fill line with a fill volume of almost 40% of the GS reservoir 11, and would be acceptable for some applications, however, given an angle between the two points and the top edge of chip 10, an equal offset from chip centre to the left (bottom left of box a) produces fill line 20f, which would be undesirable in every way: it affords a very small volume of volatile liquid (less than 10%) that would require replenishment very quickly; it provides a relatively small free surface to interact with a carrier gas stream, leading to limited entrainment; the free surface contracts dramatically with change in volume of the contained liquid; and the free surface is not advantageously positioned with respect to the carrier gas stream to strip gas produced. As a result the functional spatial optimization of this GS reservoir 11, for this reference axis position, is poor. The reference axis position for 20f is the bottom left corner of box a. A curve, c1, is drawn over boxes a,b,c that roughly delimits axis positions like that of fill line 20f, which do not admit of at least 40% fill volume relative to reservoir 11's capacity, from those that do.

FIG. 3 schematically illustrates a chip designed for operation with on an articulated blade platform, such as taught in Applicant's co-pending WO 2015/181725 and the prior art thereof, is shown in FIG. 3. The variant of FIG. 3 differs from FIG. 1 in that A) ports P1-P5 are centred on their respective chambers to avoid ejection of liquid content when the chip is tilted by 25° to −35°; B) GS reservoir 11 has a shape, and entrances to P1 and channel 16, that requires a very high positive or negative tilt to obstruct either opening for the gas flow therethrough; C) the arrangement of reservoirs are centrifugal canonical, in that the chambers that feed are axis-proximal their fed chambers; D) channels have with syphon segments for tilt angle-selective dispensation.

Regarding B), in order to ensure that the liquid from GS reservoir 11 never occludes the openings to P1 or channel 16, a shape of the GS reservoir 11 has a narrow top end for these two openings, and a larger belly for the liquid. This arrangement permits the two openings to be closer together, which independently reduces an angular variation over which one of the openings is blocked by a given volume of liquid. Consequences of occlusion of the opening to P1 are less than those of occlusion of channel 16, as a distance and direction of travel (initially against centrifugation) of a liquid plug may be less, and P1 is closer to the pressurized carrier gas supply than channel 16. It is still preferable to avoid occluding the opening to P1.

While bringing the two openings close together might look like an arrangement that invites a short path between these openings that doesn't entrain as much gas, in fact circulation of the gas within the chamber is encouraged by this design with a simple convection pattern that is expected to produce good entrainment, as opposed to other designs that bring about a larger number of circulating paths.

Regarding C), axis-proximal segments of the channels 17,18,19 are closer to axis than axis-proximal edges of their respective connected chambers (12-15) by a respective designed amount required for tilt-angle selective dispensation. The axis, as inferrable from fill lines 20 shown in GS reservoir 11 and CC 13, is presumed to be parallel to the top edge of chip 10 for the illustration (although the chip could be used with a variety of axis positions). Tilting the chip about axis 25 (shown in a top right corner of chip 10, but is typically off chip, and generally in the same area as delimited for the reference axis position (strip 22, or box a,b,c of FIG. 2)), but preferably distanced from the reference axis position, or orthogonal to it as shown. By raising or lowering the axis-proximal segments of the channels, and shapes, positions and fill volumes of the chambers, the tilt angles at which fluid transfer happens can be varied to suit control over the articulation mechanism, and to provide a protocol that is robust and reliable. By selecting the axis-proximal segments of the channels, an angular spacing between the angle ranges can be provided to avoid overlap and concurrent transfer between two or more chambers. That said, concurrent transfer may be desired, for example to avoid over-filling of CC 13. The tilting may be provided by varying a rotation speed of the chip 10, if the axis of rotation is parallel (and spatially distanced from) tilt axis 25.

As shown in FIG. 3, the chip 10 is in a reference tilt orientation, with centrifugal force directed perpendicular to the fill lines 20, as shown in an arrow from the tilt axis 25. A tilt angle of about −30° to −35° (angular range 25a) for a period of time necessary to prime the channel 19, is effective to produce a transfer of contents of CC 13 the into supernatant reservoir 14. When the chip is at the tilt angle for transfer, about ⅔ of the volume is present at or above the opening in CC 13 to reservoir 14, and this fraction is completely dispensed. Similarly a tilt angle range 25b of about 20-25° from the reference orientation results in transfer of all remaining liquid content of CC 13 to waste 15. As such, channels 18,19 are low hydrodynamic resistance, non-capillary, flow channels.

As per D), channel 17 is modified with respect to the variant of FIG. 1 to contain two parts: a syphon structure 17a and a large diameter segment 17b. By providing a large step in hydrodynamic diameter within SUP channel 17, it is possible to discretize flow, and to facilitate partial transfer from SUP reservoir 12 to CC 13. By tilting by a minimum angle of 5-15° (which depends on instant fill volume of reservoir 12) for a period sufficient to prime the syphon segment 17a, liquid will be dispensed. By limiting a flow rate through syphon structure 17a, the flow can be made gradual. By returning the tilt angle below the minimum angle of range 25c, the transfer stops. By repeating the tilting at desired intervals, additional buffer, reagent or other supply, can be supplied to the CC 13 in accordance with a desired protocol. Placement of an overfill port in CC 13 at an associated angle range, can prevent over filling of the CC 13, without risk of obstructing P3, or leakage during transfer to OUT reservoir 14. Note that this variant channel 17 can also be used with pneumatic control in the embodiment of FIG. 1 instead of the higher finesse control over pneumatic port P2, instead of valve 21, shown in FIG. 2.

As such, in use, with (e.g. cell culture media) is loaded through P2 into SUP reservoir 12, the biological sample in the CC 13, and volatile liquid in GS reservoir 11, the chip can be centrifuged at the reference angle, and then tilted during centrifugation in positive or negative angles. Each time the tilt angle is raised above the angle range 25c, and maintained long enough to prime the channel segment 17a, a volume is dispensed that depends primarily on the duration the angle is maintained. Each time the tilt angle is lowered below range 25a, a volume above a minimum level is dispensed as supernatant to OUT reservoir 14. Centrifugation will typically result in sedimentation of the biological sample to the volume below the minimum level. Finally all liquid from CC 13 can be transferred to waste 15 through siphon channel 18 by tilting the cartridge at or beyond range 25b.

Preferably any thermal control elements that are off-chip remain aligned with the respective heating volumes throughout chip tilt, such that incubation or other thermally controlled processes need not halt for fluid transfer steps. As such any off-chip metallic or like material for thermal control, may be provided on a chip holder or cartridge that tilts with the chip.

FIG. 4 is a schematic illustration of a variant chip 10 designed for operation with a centrifugal platform, for example with a pneumatic slip-ring for supplying pressure to only P1. All ports P2-5 are simply used as loading ports and vents. Chip 10 of FIG. 4 differs from that of FIG. 1 in that: no second OUT reservoir 14 is provided, and a passive, hydrodynamic metering and incremental fluid transfer system is used in place of SUP reservoir 12. The passive metering system is taught in Applicant's patent disclosure WO 2013/003935, and comprises a large volume reservoir 12a, coupled to a metering chamber 12b via a hydrodynamic resistive channel 17a′. Channel 17a′ provides a slow transfer that varies little even with substantial changes in centrifugation rate, and is designed for a specific viscosity, contact angle, and density of a supplied fluid. This fluid accumulates in metering chamber 12b at a fixed rate. Once the chamber 12b is filled to a threshold level, the siphon channel 17b′ is primed, and the fluid in chamber 12b is ejected into CC 13 all at once. Channel 17b′ may have negligible hydrodynamic resistance and ejection is a very fast process, or alternatively may have a hydrodynamic constriction near its opening to CC 13, such that liquid is introduced slowly into CC 13. This can be preferable to avoid abrupt chemical or temperature changes, for example, if the chamber 12b is not heated along with CC 13. If a constriction is provided at the opening of 17b′ and CC 13, the constriction must have substantially shorter delivery delay than channel 17a′, or else the chamber 12b provides no function. A continuous drip supply can be provided from large volume reservoir 12a directly to CC 13.

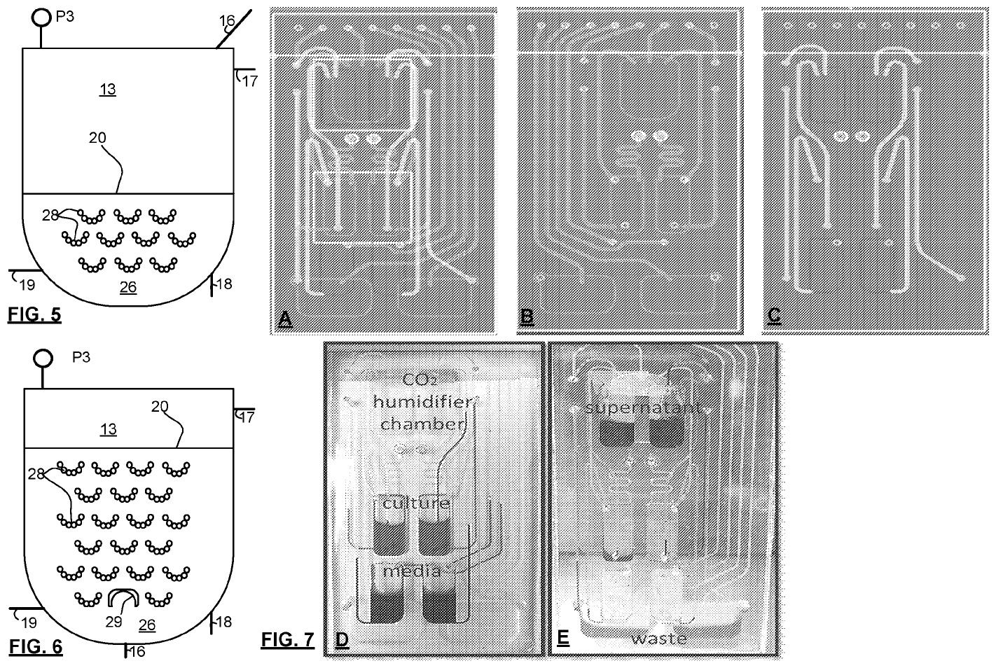

FIG. 5 is a schematic illustration of the CC 13 of the embodiments of FIGS. 1-4 in which a plurality of cell support structures 28 are provided. The cell support structures 28 are preferably microfabricated pillars formed in the same chip 10, and may be porous, and functionalized, or coated to promote cell attachment in a manner well known in the art.

FIG. 6 is a variant of the CC 13 in which the GS channel 16 meets the CC 13 from a bottom of the CC 13, to supply the carrier gas and entrained gas from the GS reservoir 11 to the CC 13 in a bubbled manner. The CC 13 may have a gas trap 29 for collecting the bubbles, and shielding the cell support structures 28 from vortices produced by bubbling, or for distributing the bubbles more uniformly across the CC 13. If a delivery rate of the carrier gas is low enough, collected gas in the gas trap 29 may dissolve before the trap overflows, providing a high efficiency gas delivery to the liquid content 26.

### EXAMPLES

Applicant has designed and tested a “biocompatible' polymer chip with all the necessary functionalities for a cell culture devices. The chip achieves biocompatibility without any functionalization or coatings. Gaseous exchange is provided by a GS reservoir, and a cell culture conditioned chamber was provided with a controlled, humidified atmosphere (controlled gaseous micro-environment). This demonstrates the use of thermoplastic polymers for device fabrication which are usually gas impermeable, and avoids a reliance upon PDMS and TPEs, while creating gas exchange conditions. The invention is demonstrated with a chip design and use. The demonstration leverages the capacities of Applicant's WO 2015/132743 to bring liquid and gases into the culture chamber and perfuse/mix them without disturbing the cells. A reliable micro-scale incubation chamber is effectively produced, allowing for long-term cell culture on a microfluidic chip, without placing the chip in an incubator. An eight port microfluidic chip controller was used to demonstrate the cell culturing on the chip. Such a controller can be adapted to provide a host of other unique functionalities that are currently not available with other cell incubation platforms: including a precise control of shear stress exerted on the cells during culture through a combined application of centrifugal and pneumatic forces. Finally, the culture chip can readily be developed with further chambers and channels for complete assay integration, including sample preparation, such as cell isolation from blood and other clinically relevant samples.

The application of the proposed platform has been demonstrated for automated culture and conditioning (activation/stimulation) of Periferal Blood Mononuclear Cells (PBMCs) such that Interferon Gamma (IFNy) released from the cells can subsequently be characterized within the context of infectious disease diagnosis applications such as Latent Tuberculosis Infection (LTBI). For this purpose, we have designed, fabricated and tested a microfluidic chip operated by the centrifugal microfluidic chip controller that has the ability to: (1) isolate and culture PBMCs in two separate chambers; (3) stimulate and incubate PBMCs with mitogen for six hours; and (3) separate the conditioned media for subsequent connection to the assay.

The microfluidic cartridge shown in FIG. 7, which is a panel of 5 elements. FIGS. 7A,B,C respectively are layouts of two layers of the chip fabricated and tested. Both top (7B) and bottom (7C) surfaces of the chip were patterned, and the patterns were interconnected by through-holes (vias), as shown in composite layout view (7A). The chip was operated to provide two cell culture chambers (CCs) with a continuous perfusion of CO₂ and prevent evaporation (by hydration) during 6 hours culture and stimulation, from a CO₂/humidifier chamber (GS reservoir). Peltier heating elements were integrated on a chip support and used to maintain the temperature of the cell culture and CO₂ humidifier chambers to 37° C. The chip was supplied, throughout centrifugation, a 5% CO₂ carrier gas stream from the chip controller. A CO₂ supply line was connected to a pneumatic pump of the chip controller and the CO₂ was continuously pumped to the heated humidifier chamber (GS reservoir containing water), allowing perfusion to the appropriate culture chambers within the chip. The cartridge also comprises separate culture media chambers (SUP reservoirs), coupled to respective CCs, as well as supernatant and waste chambers.

The microfluidic cartridge was fabricated in COC thermoplastic polymer using CNC micromachining. Applicant notes that depending on the volume requirements of the particular application, the chip is likely to be fabricated from a biocompatible thermoplastic polymer using hot embossing or injection molding, allowing for etching of additional structures within the culture chambers, such as cell traps. The chambers and channels were etched on both sides of the chip and sealed using flat COC substrates through thermal bonding (although adhesive, or solvent bonding were considered) to form a cartridge.

Photograph 7B shows two cell culture CCs, each with respective ports with serpentine paths between ports and the CCs, and each with channels (that have no constriction or valve) to a common humidifier chamber (GS reservoir) positioned above the CCs and their ports. Each of these channels passes closer to a reference axis position of the chip than either the CC or the GS reservoir. The humidifier chamber has a single port which was used both to load the chamber with water, and then as the carrier gas supply port. Photograph 7C shows respective supernatant (OUT reservoirs) and media culture (SUP reservoirs) for respective CCs.

The cartridge was mounted to the chip controller, and operated as follows. The cell culture CCs are first filled with the PBMCs suspended in their respective media, and one culture media chamber (SUP reservoir) was filled with the media supplemented with mitogen (PHA, 50 mM) and the other was filled only with cell culture media. The humidifier chamber (GS reservoir) was filled with water and the cartridge placed on the chip controller for cell culture and stimulation. The platform was centrifuged at high rotational speed (500-700 RPM) first to isolate the cells from the sample by sedimenting all the cells to a bottom of the CCs. Following initial centrifugation, the supernatant is removed to the waste and the media is replaced with fresh media for control and media with mitogen for stimulation. Throughout stimulation centrifugation at (˜300 RPM) speed and heating at 37° C. is performed under continuous 5% CO₂ perfusion. This lasted six hours. Following the stimulation, the cells are centrifuged again at high frequency and the supernatant containing stimulated cell release as well as control are moved by applying a pressure to their respective supernatant chambers for subsequent analysis.

The supernatant was analyzed using ELISA kit to measure the concentration of released IFNγ and compare the results to those obtained using standard plate culture. The six hour culture with only cell media produced an average IFNγ concentration of 45 pg/ml for the microfluidic cartridge, which is slightly lower than 67 pg/ml obtain using a standard plate. The difference may be due to suppressed cellular function due to constant rotation during the six hour experiment. Nevertheless, the obtained results indicate successful implementation of automated cell culture and stimulation assay which allows for the potential downstream integration of analytical sensors for measurement of IFNγ in the extracted supernatant. The cells were clearly unaffected by dehydration or lack of CO₂ perfusion.

Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

1. A centrifugal microfluidic chip comprising:
a plurality of microfluidic chambers each having a volume of 0.1 μL to 1 mL, and a nominal fill line defined with respect to a reference axis position;
a plurality of microfluidic channels interconnecting respective chambers;
a plurality of ports, each port in fluid communication with one or more chambers;
wherein one of the chambers is a conditioned chamber, connected via channels to chambers 1, 2 and 3, and to at least one port, where:
chamber 1 has a first opening from a port at or above its fill line, and second opening to a first channel, the first channel connecting the chamber 1 below its fill line to the conditioned chamber;
chamber 2 has a first opening from a port at or above its fill line, a second opening to a second channel at or above its fill line, and no opening to any interconnecting channel or port below the fill line, where the second channel includes a path segment that extends closer to the reference axis than any part of chamber 2, and connects the conditioned chamber above its fill line and the chamber 2 above its fill line, the second channel having no valve, and no capillary flow dimensioned constriction; and
chamber 3 has, above its fill line, a first opening to a port, and a second opening to a third channel, the third channel connecting the chamber 3 to the conditioned chamber below the conditioned chamber's fill line.

2. A centrifugal microfluidic chip comprising:
a plurality of microfluidic chambers each having a volume of 0.1 μL to 1 mL;
a plurality of microfluidic channels interconnecting respective chambers;
a plurality of ports, each port in fluid communication with one or more chambers;
wherein one of the chambers is a conditioned chamber, connected via channels to chambers 1, and 2, and connected directly to at least one port, where:
chamber 1 has a first opening from a port above a fill line of chamber 1, and second opening to a first channel, the first channel connecting the chamber 1 closer to an axis distal point of chamber 1 than its fill line, to the conditioned chamber above the conditioned chamber's fill line; chamber 2 has a spillway opening delimiting a fill line for the chamber 2, a first opening from a port above the fill line, a second opening to a second channel above the fill line, and no opening to any channel below the fill line; and
the second channel includes a path segment that extends closer to a reference axis position for the chip than either the conditioned chamber, or chamber 2, and connects to the conditioned chamber.

3. (canceled)

4. The chip according to claim 1, wherein the reference axis position of the chip:
lies within a region above the chambers, and less than 3/2 L from a top edge of the chip, and less than 0.5 L from a centre line of the chip, where L is the chip's length;
lies within a region above the chambers, and less than L from a top edge of the chip, and less than 0.5 W from a centre line of the chip, where L is the chip's length, and W is the chip's width;
lies between two lines passing through a midpoint of the two openings of chamber 2, the two lines being bisected by a perpendicular bisector of the two openings, and respectively making an angle of 30° and −30° with the perpendicular bisector; or
is separated from P1 and from the opening to the second channel, and these separations are different by no more than a factor of 2, and the fill line of chamber 2 includes a volume of at least 33% of a volume of chamber 2 below the fill line.

5.-8. (canceled)

9. The chip according to claim 1 wherein:
a surface area of the fill line in the chamber 2 is greater than that of chamber 1, by a factor of at least 2;
at least one of the conditioned chamber and chamber 2 has an etch depth greater than that of any other chamber or channel of the chip; or
a shape of the chamber 2 results in a change in free surface area of a liquid content thereof of less than 10% with a 10% decrease in volume of the liquid content from the fill line.

10.-11. (canceled)

12. The chip according to 1 wherein the conditioned chamber comprises:
a plurality of openings to outlet channels at different axial distances beyond the fill line to extract different centrifugal fractionates;
a support for a microorganism or a cell; or
a gas trap, collinear with the opening to the second channel and reference axis position, with the gas trap located between the reference axis position and the opening, for retaining gas bubbles while the gas diffuses into the liquid.

13. The chip according to claim 1 wherein a subset of the ports are provided for addressable pneumatic actuation, and these ports are aligned along an edge of the chip for concurrent clamped sealing connection; or the port of chamber 2 has a form suitable for coupling to a sealed supply tube.

14.-15. (canceled)

16. The chip according claim 1 wherein a thermally absorbing and distributing material is provided adjacent one of the conditioned chamber and the chamber 2.

17. The chip according to claim 16 wherein two separately addressable bands of the material are provided for independently heating the conditioned chamber, and the chamber 2.

18. The chip according to claim 1 wherein the chip is covered and forms a microfluidic cartridge adapted for mounting to a centrifuge with at least one pneumatically addressable port of the chip coupled to a pressurized carrier gas supply of the centrifuge.

19. The chip according to claim 1 wherein the second channel:
meets the conditioned chamber at an opening below its fill line; or
is subject to free flow with negligible capillary effects and hydrodynamic resistance, and has neither a hydrophilic nor a hydrophobic coating.

20. (canceled)

21. The chip according to claim 1 wherein each chamber 1 is positioned axis-proximal each conditioned chamber, and any chamber 3 is positioned axis-distal each conditioned chamber.

22. The chip according to claim 1 wherein the chip is composed of a thermoplastic, thermoplastic elastomer, or PDMS with a suitable gas impermeable layer.

23. The chip according to claim 1 wherein the chip is composed of a thermoplastic elastomer layer, and at covering layer defining a pneumatic control layer, and one or more ports of the chip are used to actuate pneumatic valves within the chip, each valve being a respective one of the following: a normally closed valve, a normally open valve, or a tristate valve with open, closed, and semipermanently closed states.

24. (canceled)

25. The chip according to claim 1 wherein three or more of the channels other than the second channel, have respective axis-proximal segments, and connect the conditioned chamber to respective chambers having shapes, positions, and fill lines selected so that a tilt angle of the chip about a tilt axis perpendicular to the surface of the chip, permits fluid transfer between the conditioned chamber and only one of these chambers.

26. The chip according to claim 25 wherein a range of tilt angles of less than 45° is sufficient to sequentially transfer between the conditioned chamber and the three respective chambers.

27. The chip according to claim 1 wherein the first channel comprises a hydrodynamic constriction and a metering chamber.

28. A kit comprising the chip according to claim 1, accompanied by one or more of:
a liquid for loading onto the chip;
one or more cartridge forming elements that combined with chip form cartridges that are adapted for coupling to one of a centrifugal microfluidic chip controller; a centrifuge blade with a pneumatic slip ring; or an articulated centrifuge blade with a pneumatic slip ring; and
a material for application to a cartridge, the chip, or a chip support, the material having a composition and dimension to provide thermal control over the conditioned chamber; the chamber 2; or a part of one of these below their fill line.

29. The kit according to claim 28 wherein the liquid comprises one of: volatile liquid content for the chamber 2; a liquid containing or potentially containing a biological sample for the conditioned chamber; or one or more reagents, buffers, or solutions for chamber 1.

30. The kit according to claim 28 wherein the kit is assembled and mounted to the centrifugal microfluidic chip controller; centrifuge blade with a pneumatic slip ring; or articulated centrifuge blade with a pneumatic slip ring.

31. A centrifugal microfluidic system comprising the chip of claim 1, with a liquid contained in one of the chambers, wherein the liquid contained in one of the chambers is: a volatile liquid content for the chamber 2; a liquid containing a biological sample in the conditioned chamber; or one or more reagents, buffers, or solutions in chamber 1.

32. (canceled)
